(19) United States
(12) Patent Application Publication
(10) Pub. No.: US 2011/0160608 A1
(43) Pub. Date: Jun. 30, 2011

(54) Brain-related chronic pain disorder diagnosis and assessment method

(75) Inventor: Jeffrey B. Hargrove, Bancroft, MI (US)

(73) Assignee: CEREPHEX CORPORATION, Bancroft, MI (US)

(21) Appl. No.: 12/809,109

(22) PCT Filed: Dec. 18, 2008
(86) PCT No.: PCT/US08/87451

§ 371 Date: Jun. 18, 2010

Related U.S. Application Data
(60) Provisional application No. 61/014,917, filed on Dec. 19, 2007.

(51) Int. Cl. A61B 5/0476

(52) U.S. Cl. 600/544

(57) ABSTRACT

A method for diagnosing and assessing a brain-related chronic pain disorder. The method includes assessing a subject's brain function, determining the probability that a subject is suffering from chronic pain as a result of an abnormal brain function condition by obtaining a quantitative assessment of the subject's brain function, and making a statistical comparison between the subject's quantitative brain function assessment and either a database of quantitative assessments of the brain functions of normal, healthy individuals, or a database of quantitative assessments of the brain functions of individuals known to have been suffering from chronic pain as a result of the abnormal brain function condition.

### CROSS-REFERENCES TO RELATED APPLICATIONS

This application claims priority in U.S. Provisional Patent Application Ser. No. 61/014,917, filed Dec. 19, 2007.

### BACKGROUND OF THE INVENTION

1. Field of the Invention

This invention relates generally to a method for diagnosing and assessing brain-related chronic pain disorders in human subjects.

2. Description of Related Art

Several methods of diagnosing and assessing fibromyalgia are known in the art. For example, U.S. Pat. No. 5,834,215 issued 10 Nov. 1998 to Garry, et al. discloses a method of detecting antipolymer antibodies as a means of diagnosing fibromyalgia. U.S. Pat. No. 7,056,686 issued 6 Jun. 2006 to Lin, et al., discloses a method for diagnosing fibromyalgia by detecting the presence of small intestinal bacterial overgrowth. In addition, U.S. Pat. No. 7,146,205 issued 5 Dec. 2006 to Holman teaches a diagnosis and treatment method involving the use of inhibitors of sympathetic nervous system activities. However, none of these methods address the role of brain function in the pathology of fibromyalgia.

Also known in the art are methods for quantitatively analyzing analog electroencephalogram (EEG) signals. These methods are generally known as “quantitative electroencephalography”, or qEEG, and are disclosed, for example, in U.S. Pat. No. 5,230,346 issued 27 Jul. 1993 to Leuchter, et al. and U.S. Pat. No. 6,097,980 issued 1 Aug. 2000 to Monastra, et al. However, nothing in the prior art of record contemplates the use of qEEG analysis methods to diagnose or assess a brain-related chronic pain disorder such as fibromyalgia.

In addition, the use of EEG and qEEG analyses in diagnosis, assessment, prognosis, and therapeutic activities is known in the art and disclosed, for example, in U.S. Pat. Nos. 7,276,026 issued 2 Oct. 2007 to Skinner; U.S. Pat. No. 7,177,675 issued 13 Feb. 2007 to Suffin, et al.; U.S. Pat. No. 7,269,455 issued 11 Sep. 2007 to Pineda; U.S. Pat. No. 7,231,245 issued 12 Jun. 2007 to Greenwald, et al.; U.S. Pat. No. 6,097,980 issued 20 Sep. 2005 to Gevins, et al; and U.S. Pat. No. 6,947,790 issued 20 Sep. 2007 to Gevins, et al. However, none of the diagnostic and assessment methods disclosed in the '026, '675, '455, '245, '980, and '790 patents is able to diagnose or assess brain-related chronic pain disorders such as fibromyalgia.

What is needed is a method for diagnosing and assessing brain-related chronic pain disorders in human subjects by assessing brain function.

### BRIEF SUMMARY OF THE DISCLOSURE

A method is provided for diagnosing and assessing a brain-related chronic pain disorder. The method includes the steps of assessing a subject's brain function, determining the probability that a subject is suffering from chronic pain as a result of an abnormal brain function condition by obtaining a quantitative assessment of the subject's brain function, and making a statistical comparison between the subject's quantitative brain function assessment and either a database of quantitative assessments of the brain functions of normal, healthy individuals, or a database of quantitative assessments of the brain functions of individuals known to have been suffering from chronic pain as a result of the abnormal brain function condition.

### BRIEF DESCRIPTION OF THE DRAWINGS

These and other features and advantages of the invention will become apparent to those skilled in the art in connection with the following detailed description and drawings, in which:

FIG. 1A is a flow chart depicting a method performed according to the invention;

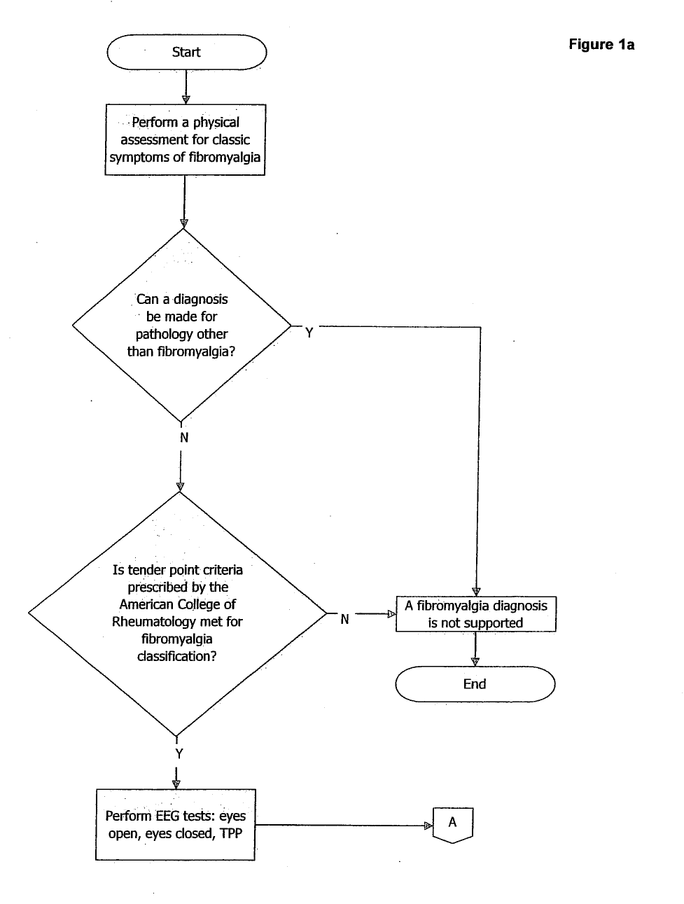

FIG. 1B is a continuation of the flow chart of FIG. 1A;

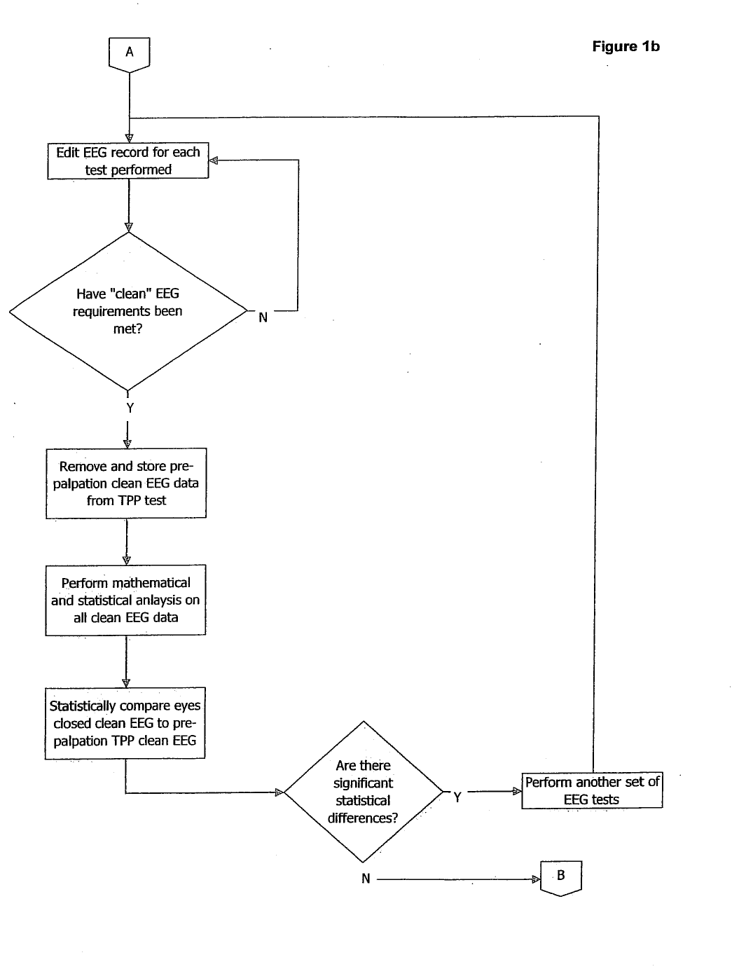

FIG. 1C is a continuation of the flow chart of FIG. 1B;

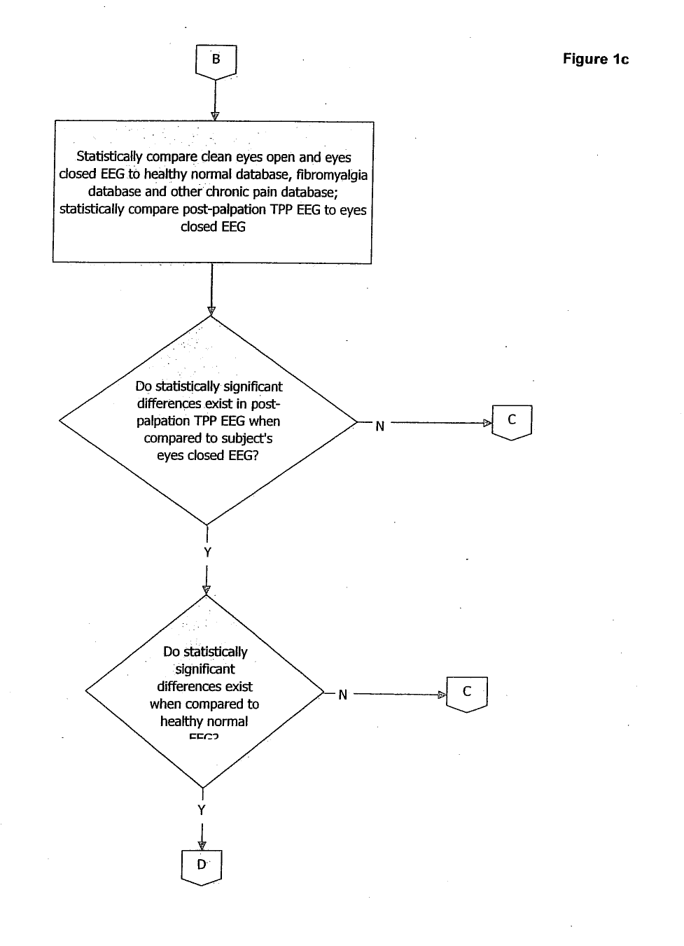

FIG. 1D is a continuation of the flow chart of FIG. 1C; and

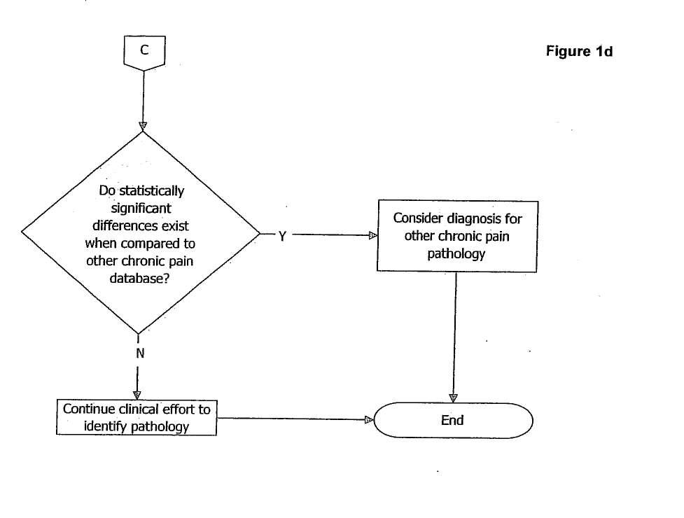

FIG. 1E is a continuation of the flow chart of FIG. 1D.

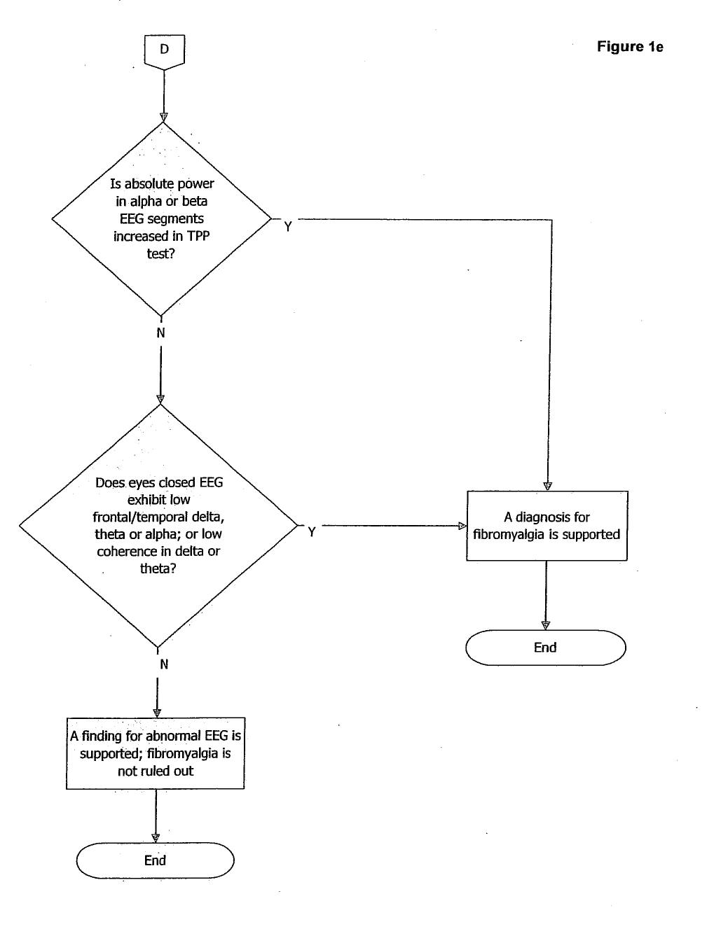

### DETAILED DESCRIPTION OF INVENTION EMBODIMENT(S)

A method is provided for diagnosing and assessing a brain-related chronic pain disorder. The method includes assessing a human subject's brain function and then determining the probability that the subject is suffering from chronic pain related to an abnormal brain function condition by obtaining a quantitative assessment of the subject's brain function and making a statistical comparison between the subject's quantitative brain function assessment and a database of quantitative assessments of the brain functions of individuals known to have been suffering from chronic pain as a result of the abnormal brain function condition. The assessment of a subject's brain function may include obtaining an electroencephalogram (EEG) of the subject's electrical brain activity, and the determination of the probability that the subject is suffering from chronic pain as a result of an abnormal brain function condition may include determining the probability that the subject is suffering from a chronic pain condition such as fibromyalgia by obtaining a quantitative assessment of the subject's EEG (qEEG) and making a statistical comparison between the subject's qEEG and a database of qEEGs of individuals known to have been suffering from fibromyalgia.

A physical assessment may first be performed of a human subject presenting with a complaint of symptoms characteristic of a chronic pain condition such as fibromyalgia. The physical assessment may include, among other things, a determination of chronic widespread pain, sleep difficulty, fatigue, morning stiffness of the muscles and joints, cognitive difficulty and other symptoms associated with the condition. Where, for example, fibromyalgia is suspected, the physical assessment may also include tests performed to exclude various non-fibromyalgia conditions as the cause of the symptoms. Such further testing may include palpation of 18 tender points in the manner prescribed by the American College of Rheumatology (ACR), with such palpation being performed to determine whether the subject has an abnormal sensitivity to pain. Where, for example, idiopathic chronic low back pain ICLBP) is suspected, the physical assessment may include tests performed to exclude various non-ICLBP conditions as the cause of the symptoms. Such further testing may include palpation of tender points other than the 18 tender points prescribed by the ACR and/or may include physical tests other than tender point palpation.

In the absence of a definitive diagnosis, an EEG test may be performed in addition to the physical assessment. Specifically, the subject may be made comfortable by, for example, being seated or reclined. Preparation of the scalp in accordance with commonly followed procedures for performing a clinical EEG may be done by a person of sufficient competence. EEG electrodes may then be adapted to be worn on the scalp, preferably in scalp locations identified as the “International 10-20” standard sites, using common methods of affixing the electrodes such that they rest on or otherwise contact tissues.

While any number of electrodes may be used, a preferred number is either 19 or 24, in accordance with the number of electrode sites used to construct various independent databases utilized to represent the EEG of a healthy normal population.

Records of the subject's EEG from each electrode site may then acquired under the conditions of both their eyes being closed and their eyes being open, with each condition producing a separate data record. In other words, an “eyes open” EEG record may be obtained, which includes EEG data obtained from each electrode site while the subject's eyes are open and an “eyes closed” EEG record may be obtained, which includes EEG data obtained from each electrode site while the subject's eyes are closed. Preferably, a minimum of five minutes of EEG data may be obtained from each electrode site for each “eyes open” EEG record and a minimum of five minutes of EEG data may be obtained from each electrode site for each “eyes closed” EEG record to assure that enough EEG data is recorded to produce statistically significant samples from each electrode site, both with the subject's eyes open and with the subject's eyes closed. This is further described below.

Preferably, an additional test may be performed in which at least one additional EEG record is made that includes EEG data obtained at each electrode site while pain is elicited in the subject. In diagnosing or assessing conditions such as fibromyalgia, a number of tender points on the subject's body may be palpated. In this test, henceforth referred to as a “tender point palpation (TPP) test”, a number of tender points on the subject's body, preferably ranging between one and 18 when diagnosing or assessing fibromyalgia, are identified and serially palpated, preferably with an algometer. Preferably, four tender points may be chosen, and, preferably, those four points include tender points adjacent the right and left lateral epicondyle of the arms, and tender points adjacent the right and left costochondral junctions of the second rib. While the subject's eyes are preferably closed during this test, it should not be confused with the “eyes closed” test described above.

The TPP test may be executed by acquiring an EEG record (“TPP” EEG record) including EEG data obtained from the electrode sites for a first tender point by first commencing the acquisition of EEG data and then, a short period of time later, commencing palpation of the first tender point. Preferably, the period of time between the commencement of data acquisition and the commencement of palpation of the first tender point may be between one and three hundred seconds. Palpation of the first tender point may be accomplished by pressing on the tender point with an algometer, preferably at a rate of approximately one kilogram per centimeter squared per second, until the subject reports a painful sensation. Preferably, palpation pressure may be removed as soon as the subject reports a painful sensation. A record is made of the amount of the pressure being applied at the moment the subject reports a painful sensation. Although the TPP EEG record may be obtained while the subject's eyes are closed, it should not be confused with the “eyes closed” EEG record described above.

Further according to the TPP test method, the acquisition of the TPP EEG record may include continued recording of EEG data (with the subject's eyes closed) for a period of time after release of palpation pressure, preferably between 1 and 300 seconds, and most preferably, for at least 60 seconds. A comparison may then be made between EEG data collected before application of palpation pressure and EEG data collected after release of palpation pressure. This comparison may then be used to make diagnostic findings. Such findings may include changes in brain EEG activity, when comparing EEG after release of palpation pressure to EEG before palpation pressure, in specific regions of the brain characteristic of a brain-related chronic pain condition, but not otherwise anticipated in a healthy normal individual.

Following this period, a second and subsequent tender point may be serially palpated, preferably with an algometer, in the same manner as described for the first, with TPP EEG records being recorded for each by recording the eyes closed EEG for each site in the manner described with regard to obtaining the TPP EEG record for the first site. This process may be repeated for each chosen tender point. Accordingly, the resulting EEG data record includes the TPP EEG records acquired for each chosen tender point.

The “TPP” EEG records may be acquired for a period of time that is sufficient to extract from each “TPP” EEG record a minimum of 60 seconds of “clean” EEG data, that is, data free of extraneous electrical noise such as that from electromyographic movement. Preferably, all EEG records (“eyes open” EEG records, “eyes closed” EEG records, and “TPP” EEG records) may be individually edited to provide from each EEG record a minimum of 60 seconds of clean EEG. Preferably, the clean data is obtained so as to present a high degree of statistical consistency. Such measures as “Split-Half” reliability, which is the ratio of variance between the even and odd seconds of the time series of selected clean EEG; and “Test Re-test” reliability, which is the ratio of variance between the first half and the second half of the selected clean EEG segments may be used. Preferably, clean EEG data is obtained such that measures of these ratios are a minimum of 0.95 and 0.90 respectively, which is consistent with levels of reliability commonly published in EEG literature.

With regard to the TPP test method, clean data includes that EEG data acquired after palpation of a tender point, and does not include any EEG data acquired during the palpation of a tender point. In addition, to assess the stability of a TPP EEG record, EEG data acquired before palpation of a tender point may be removed, edited and statistically compared to like data in the “eyes closed” EEG record obtained from the eyes closed EEG test. Stability of the “closed eyes” and TPP EEG records is indicated by a finding that there is no statistically significant difference between the “eyes closed” EEG record and the pre-palpation portion of the. TPP EEG record. A contrary finding indicates instability and a need to repeat the EEG tests.

Further to the method, and in the preferred embodiment, clean “eyes open”, “eyes closed”, and “PPT” EEG records may be then mathematically analyzed for various time domain and frequency domain parameters of their respective electrical signals. These analyses may include, but are not limited to voltage and current analyses, frequency spectrum analyses using methods such as a Fast Fourier Transform or wavelet analysis, an absolute power analysis, a relative power analysis, a phase analysis, a coherence analysis, an amplitude asymmetry analysis, and localization of electrical activity in the brain using inverse EEG computation analysis.

Findings from the aforementioned analyses may then be statistically compared to the same parameters determined from “eyes open”, “eyes closed”, and “PPT” EEG records taken from an age and gender matched database of healthy normal individuals. Such statistical analyses may include, but are not limited to deviations from a standard normal distribution. Findings of statistically significant abnormal deviation, or lack thereof; may then be presented in a graphical or numerical format for analysis by a competent health care professional or person of similar expertise.

EEG abnormalities consistent with those observed in a sample population of fibromyalgia patients may include, but are not limited to one or more of the following: (1) an overall reduction in EEG power across all spectra in either of the eyes open or eyes closed conditions; (2) statistically significant low EEG power levels in frontal or temporal regions of any of the delta (1-3.5 hertz), theta (4-7.5 hertz) or alpha (8-12 hertz) frequency segments of EEG for the eyes closed condition; (3) statistically significant low coherence among the frontal EEG sites for the delta or theta EEG segments in either of the eyes closed or eyes open conditions; (4) statistically significant high relative beta (12.5-25 hertz) absolute power in the parietal region of the brain for either of the eyes closed or eyes open conditions. The magnitude of statistical variation considered to be statistically “significant” may vary depending on the application. For example, in research, a difference between a sample and a population measure generally has to have a p-value of 0.01 or less for the difference to be considered statistically “significant”. However, in clinical application statistically significant differences may be declared with p-values at the 0.1 level or less.

Further EEG abnormalities consistent with those observed in a sample population of fibromyalgia patients, and drawn particularly to the TPP test method, may include but are not limited to a finding of (1) a statistically significant increase in EEG absolute power, particularly in the alpha and beta segments, in the parietal and occipital areas of the brain as compared to the “eyes closed” EEG record (“eyes closed” EEG findings without tender point palpation) for the same subject; or (2) a statistically significant increase in coherence in the alpha or beta segment of EEG.

A diagnosis of fibromyalgia may be made when physical assessment findings that support a diagnosis of fibromyalgia are augmented by (1) at least one abnormal finding resulting from the TPP test, preferably a finding of a statistically significant increase in EEG absolute power, and particularly in the alpha and beta segments, in the parietal and occipital areas of the brain as compared to the eyes closed findings without tender point palpation for the same subject; and preferably (2) at least one abnormal finding resulting from the eyes closed EEG test, preferably statistically significant low EEG power levels in frontal or temporal regions of any of the delta, theta or alpha frequency segments of EEG for the eyes closed condition, and most preferably with an additional finding of statistically significant low coherence among the frontal EEG sites for the delta or theta EEG segments.

Clean EEG records from a subject may be mathematically analyzed for various time domain and frequency domain parameters of their electrical signals, consistent with analysis techniques already described, and then findings from these mathematical analyses may be statistically compared to like parameters taken from an age and gender matched database of individuals known to have fibromyalgia. The statistical comparisons may include, but are not limited to deviations from a standard normal distribution of like EEG measures associated with members of a database of individuals known to have fibromyalgia. The results of those comparisons may then be presented in a graphical or numerical format for analysis by a competent health care professional or person of similar expertise for the existence of statistically significant abnormal deviations, or the lack thereof. A finding in support of a fibromyalgia diagnosis would be supported if there is an absence of any significant deviation between measures from a subject's clean EEG and those from a database comprising individuals known to have fibromyalgia.

Similarly, clean EEG from a subject may be mathematically analyzed for various time domain and frequency domain parameters of its electrical signals, consistent with analysis techniques already described, and then findings from these mathematical analyses may be statistically compared to like parameters determined from an age and gender matched database of individuals known to have a chronic pain condition other than fibromyalgia.

The statistical comparisons may include, but are not limited to deviations from a standard normal distribution of like EEG measures associated with members of a database of individuals known to have the chronic pain condition. The results of those comparisons may then be presented in a graphical or numerical format for analysis by a competent health care professional or person of similar expertise for the existence of statistically significant abnormal deviations, or the lack thereof. A finding in support of a chronic pain condition diagnosis would be supported if there is an absence of any significant deviation between measures from a subject's clean EEG and those from a database comprising individuals known to have the chronic pain condition.

To determine the probability that a subject belongs to a population of individuals suffering from fibromyalgia a statistical comparison may be made of EEG parameters of the subject, as determined from the aforementioned analyses, to like EEG parameters determined from a database of individuals known to suffer from fibromyalgia. The statistical comparison may include, but is not limited to, determination of z-statistics associated with specific EEG measures from a standard normal distribution determined from the database of individuals known to suffer from fibromyalgia. Probability of inclusion in the population of individuals suffering from fibromyalgia would result from findings that subject measures cannot be excluded from the database standard normal distribution. Assuming that the data in the database of fibromyalgia patient EEG is normally distributed, then statistics such as the t-statistic or the z-statistic can be used to determine the probability that the sample EEG belongs to the population of fibromyalgia sufferers. If the probability is sufficiently low (e.g. p<0.01) then a conclusion could be made that the sample does not belong to that population.

Similarly, the probability that a subject belongs to the population of individuals suffering from a chronic pain condition other than fibromyalgia may be determined by making statistical comparison of EEG parameters of a subject, determined from the aforementioned analyses, to like EEG parameters determined from a database of individuals known to suffer from that chronic pain condition. The statistical comparison may include, but is not limited to, determination of z-statistics associated with specific EEG measures from a standard normal distribution determined from the database of individuals known to suffer from the chronic pain condition. Probability of inclusion in the population of individuals suffering from the chronic pain condition other than fibromyalgia would result from findings that subject measures cannot be excluded from the database standard normal distribution.

In addition, findings from aforementioned analyses of clean EEG records from a subject may be statistically correlated to measures of symptom severity. As previously described, analysis findings may be mathematically analyzed for various time domain and frequency domain parameters of their electrical signals. A number of measures of the magnitude of deviation from standard normal distributions of either healthy normal EEG, known fibromyalgia patient EEG, or from EEG of individuals known to suffer from a chronic pain condition other than fibromyalgia can be determined. The magnitudes are presumed to be related to the severity of the condition, and may be statistically correlated to such symptom measures that may include, but are not limited to tender point pain pressure thresholds as determined by an algometer, and various other indices of pain derived from the algometry measures (e.g. the sum of all 18 tender point pain tolerance measures, the average of all 18 tender point pain tolerance measures, etc.). Such analysis has utility in both predicting symptom severity in individuals with fibromyalgia, and in determining the effect of therapeutic intervention to correct or manage symptoms of fibromyalgia.

Also the above-described EEG testing and statistical analysis methods may be repeated on a subject following a period of therapeutic intervention on the subject. The results of these statistical analyses may be statistically compared to like statistical analyses of the subject accomplished before therapeutic intervention was started. This comparison might include, but is not be limited to, paired t-testing statistics, correlation analysis of changes in symptom severity, and subsequent comparison to a database of age and gender matched healthy normal individuals. The comparisons could be used as a means of assessing the effectiveness of a chosen therapeutic intervention, or as a means of determining if an alternate intervention may be indicated in the absence of treatment effect from a current therapeutic intervention. The comparisons could also be used as a means of determining if further therapeutic intervention may be indicated in the absence of any abnormal findings. With regard to the TPP test, repeat testing may include applying tender point pressure with an algometer only to the levels required to cause a painful response recorded in the same testing performed before therapeutic intervention.

EEG data may be acquired from a subject at a first location (e.g. a clinical location) and the EEG data may be transferred via electronic means to another location (e.g. a central analysis location) for the herein described analysis and statistical comparisons. The electronic means of data transfer may include, but is not be limited to, data transfer across a local area network or the internet. Analyses and statistical findings may then be transferred from the central analysis location to the clinical location where they can be used in various ways by a physician or similarly qualified health care professional for the determination of best clinical practice and therapeutic intervention.

EEG data may also be acquired from a subject at a first location (e.g. a clinical location) and the EEG data transferred via electronic means to another location (e.g. a central analysis location) for the purpose of increasing the size of various databases of individuals known to be suffering from fibromyalgia, individuals known to be suffering from a chronic pain condition other than fibromyalgia, and/or healthy normal individuals.

In testing for chronic pain conditions other than fibromyalgia, other, more general physical tests may be performed. Some of those tests may include a form of tender point palpation that differs from that typically done in testing for fibromyalgia, and that differs in a way that makes the testing more useful in diagnosing other chronic pain conditions. For example, tests involving algometer palpation may be performed at several points on the body of a suspected ICLBP patient, but not necessarily at the same 18 tender points described above for diagnosing and/or assessing fibromyalgia. Testing for ICLBP may include some other form of tender point palpation including physical action that causes reproduction of the back pain. Just as in the method disclosed for diagnosing and/or assessing fibromyalgia, this general physical test may be done following a period of EEG collection, and then additional EEG data may be captured after the test. Further, just as in the method disclosed for diagnosing and/or assessing fibromyalgia, differences in the EEG data may then be analyzed and/or statistically compared to determine if the result belongs to a particular chronic pain condition such as ICLBP. For example, the ideal test for an ICLBP patient might include palpation of four FM tender points and performance of a number of other physical actions that cause reproduction of pain specific to ICLBP patients. If the EEG analysis then shows a negative finding for the fibromyalgia tender points but a positive finding for the back pain actions, then a conclusion that the patient has ICLBP would be supported rather than a conclusion that the patient is suffering from fibromyalgia.

This description, rather than describing limitations of an invention, only illustrates embodiments of the invention to be recited in the claims. The language of this description is therefore exclusively descriptive and is non-limiting. Obviously, it's possible to modify this invention from what the description teaches. One may practice the invention other than as described above.

1. A method for diagnosing and assessing a brain-related chronic pain disorder, the method including the steps of:
assessing a subject's brain function;
determining the probability that a subject is suffering from chronic pain as a result of an abnormal brain function condition by obtaining a quantitative assessment of the subject's brain function; and
making a statistical comparison between the subject's quantitative brain function assessment and either:
a database of quantitative assessments of the brain functions of normal, healthy individuals, or
a database of quantitative assessments of the brain functions of individuals known to have been suffering from chronic pain as a result of the abnormal brain function condition.

2. The method of claim 1 in which:
the step of assessing a subject's brain function includes obtaining an electroencephalogram (EEG) of the subject's electrical brain activity; and
the step of determining the probability includes determining the probability that the subject is suffering from the abnormal brain function condition by:
obtaining a quantitative assessment of the subject's EEG (qEEG), and
making a statistical comparison between the subject's qEEG and either:
a database of qEEGs of normal, healthy individuals, or
a database of qEEGs of individuals known to have been suffering from the abnormal brain function condition.

3. The method of claim 2 in which:
the step of assessing a subject's brain function includes obtaining an electroencephalogram (EEG) of the subject's electrical brain activity;
the step of determining the probability that a subject is suffering from chronic pain as a result of abnormal brain function includes:
obtaining a quantitative assessment of the subject's EEG (qEEG), and
making a statistical comparison between the subject's qEEG and a database of qEEGs of individuals known to have been suffering from chronic pain as a result of abnormal brain function; and
the step of determining the probability that the subject is suffering from chronic pain as a result of the abnormal brain function condition includes making a statistical comparison between the subject's qEEG and a database of qEEGs of the brain functions of individuals known to have been suffering from the abnormal brain function condition.

4. (canceled)

5. (canceled)

6. The method of claim 2 in which the step of assessing a subject's brain function includes obtaining a “TPP EEG” record of EEG data obtained over a period of time during which at least one tender point on the subject's body is palpated.

7. The method of claim 6 in which an algometer is used to palpate at least one tender point on the subject's body while the “TPP EEG” record is being obtained.

8. The method of claim 6 in which:
the step of making a statistical comparison includes making such a comparison between the subject's qEEG and a database of qEEGs of individuals known to have been suffering from fibromyalgia; and
the step of palpating at least one tender point includes serially palpating the right and left lateral epicondyle of the arms, and the right and left costochondral junctions of the second rib.

9. (canceled)

10. The method of claim 6 in which the step of palpation at least one tender point includes increasing pressure on the tender point with an algometer until the subject reports a painful sensation, and recording the amount of pressure being applied at the moment the subject reports a painful sensation.

11. (canceled)

12. (canceled)

13. (canceled)

14. The method of claim 6 in which the palpation and recording steps are repeated for each tender point.

15. (canceled)

16. (canceled)

17. (canceled)

18. The method of claim 6 in which:
the step of obtaining an EEG includes:
acquiring an “eyes closed” EEG record of data obtained while the subject's eyes are closed for a period of time sufficient to obtain statistically significant sample; and
acquiring an “eyes open” EEG record of data obtained while the subject's eyes are open for a period of time sufficient to obtain statistically significant sample; and
the step of obtaining a “TPP EEG” record includes determining stability of the EEG by comparing pre-palpation EEG data with EEG data obtained from the “eyes closed” EEG record, and repeating the step of obtaining an EEG if there is a statistically significant difference between the pre-palpation EEG data of the “TPP EEG” record and the EEG data of the “eyes closed” EEG record.

19. The method of claim 1 in which the step of assessing a subject's brain function includes obtaining information on the subject's brain using positron-emission tomography (PET), magnetic resonance imaging (MRI), or single photon emission computed tomography (SPECT).

20. (canceled)

21. (canceled)

22. The method of claim 1 in which:
the step of assessing a subject's brain function includes obtaining an electroencephalogram (EEG) of the subject's electrical brain activity; and
the step of obtaining a quantitative assessment includes obtaining one or more qEEG parameters by mathematically analyzing the subject's EEG using one or more qEEG methods selected from the group consisting of voltage analysis, current analysis, voltage and current analysis, frequency spectrum analysis using Fast Fourier transform analysis, frequency spectrum analysis using a wavelet analysis method, frequency spectrum analysis using absolute power analysis method, frequency spectrum analysis using relative power analysis method, frequency spectrum analysis using phase analysis method, frequency spectrum analysis using coherence analysis method, frequency spectrum analysis using amplitude symmetry analysis method, and localization of electrical activity in the brain using inverse EEG computation analysis.

23. The method of claim 22 in which a particular abnormal brain function condition is diagnosed as a factor related to subject's chronic pain by:
detecting one or more deviations in a statistical comparison of the subject's one or more qEEG parameters to like qEEG parameters obtained from at least one healthy normal individual; and
comparing the one or more deviations to deviations detected in individuals known to have been suffering from the abnormal condition.

24. (canceled)

25. (canceled)

26. The method of claim 1 in which the step of making a statistical comparison includes determining statistics associated with at least one EEG parameter from a standard normal distribution of like parameters in a database of individuals known to have been suffering from an abnormal brain function condition causing chronic pain.

27. (canceled)

28. (canceled)

29. (canceled)

30. (canceled)

31. (canceled)

32. The method of claim 28 in which the abnormal brain function condition being diagnosed is fibromyalgia:
the step of assessing a subject's brain function includes obtaining a “TPP EEG” record of EEG data obtained over a period of time during which at least one tender point on the subject's body is palpated; and
the deviation detected in a sample population of known fibromyalgia patents may include any one or more of the deviations included in the group of deviations consisting of a statistically significantly higher EEG absolute power values in the “TPP EEG” records taken in the parietal and occipital areas of the brain as compared to the “eyes closed” EEG records taken in the parietal and occipital areas of the brain of the same subject; and a statistically significant increase in coherence in the alpha or beta segment of the “TPP EEG” record.

33. (canceled)

34. (canceled)

35. (canceled)

36. (canceled)

37. (canceled)

38. (canceled)

39. The method of claim 2 including the additional step of providing evidence of treatment effect on the abnormal brain function condition by performing EEG measures and qEEG analyses on a subject suffering from the abnormal brain function condition following a period of therapeutic intervention on the subject and making a statistical comparison to like data obtained before therapeutic intervention.

40. (canceled)

41. The method of claim 1 in which the comparison is used to assess the effectiveness of a chosen therapeutic intervention, to determine whether an alternate intervention is indicated, and/or to determine whether further therapeutic intervention is indicated.

42. The method of claim 2 in which:
the step of assessing a subject's brain function includes obtaining a “TPP EEG” record of EEG data obtained over a period of time during which at least one tender point on the subject's body is palpated; and
the EEG measures and qEEG analyses include obtaining and comparing “TPP EEG” records from both before and after therapeutic intervention.

43. (canceled)

44. The method of claim 2 including the additional step of predicting symptom severity associated with the abnormal brain function condition by correlating at least one qEEG measure of symptom severity in a subject suffering from the abnormal brain function condition to like qEEG measures of symptom severity in a group of individuals known to be suffering from the abnormal brain function condition.

45. (canceled)

46. The method of claim 44 in which the at least one qEEG measure of symptom severity includes the magnitude of deviation from a standard normal distribution of like qEEG measures from a database of such measures taken from normal, healthy individuals.

47. (canceled)

48. (canceled)

49. (canceled)

50. (canceled)

51. (canceled)

52. The method of claim 3 in which the step of determining the probability that the subject is suffering from chronic pain as a result of the abnormal brain function condition includes making a statistical comparison between the subject's qEEG and a database of qEEGs of the brain functions of individuals known to have been suffering from one or more abnormal brain function conditions selected from the group of such conditions consisting of fibromyalgia, chronic back pain, and chronic headache.
